(19) United States
(12) Patent Application Publication
(10) Pub. No.: US 2021/0038846 A1
(43) Pub. Date: Feb. 11, 2021

(54) INTUBATION APPARATUS WITH AN ADJUSTABLE ENDOTRACHEAL TUBE AND AN ADJUSTABLE AND REMOVABLY ATTACHABLE BITE BLOCK

(71) Applicant: Matthew C. Gertsch, St. George, UT (US)

(72) Inventor: Matthew C. Gertsch, St. George, UT (US)

(21) Appl. No.: 16/989,412

(22) Filed: Aug. 10, 2020

Related U.S. Application Data
(60) Provisional application No. 62/884,876, filed on Aug. 9, 2019.

(51) Int. Cl. A61M 16/04

(52) U.S. Cl. CPC A61M 16/0493; A61M 2205/02; A61M 2205/583; A61M 2205/3327

(57) ABSTRACT

An endotracheal tube includes a removably attachable bite block. The removably attachable bite block may have a hard, inner surface for prohibiting compression of the endotracheal tube and a soft, outer surface for reducing potential damage to a patient's teeth. The removably attachable bite block is slidable on the endotracheal tube, allowing custom placement by medical staff.

### CROSS-REFERENCE TO RELATED APPLICATIONS

This application claims the benefit of U.S. Provisional Application Ser. No. 62/884,876, filed on Aug. 9, 2019, which is incorporated herein by reference.

### TECHNICAL FIELD

The present disclosure relates to bite blocks and endotracheal tubes. More particularly, the present disclosure relates to a removable and adjustable bite block and an adjustable endotracheal tube for ease of insertion.

### BACKGROUND

Intubation is an important part of modern medical care. An endotracheal tube assists a patient's breathing by being inserted through the mouth and then into the airway. Once the endotracheal tube is placed in the airway, it is then connected to a ventilator. Intubation is used to assist a patient's breathing during, for example, anesthesia or an illness. Many people throughout the world need to be intubated each year, which often requires a bite block. A bite block prevents many potential issues for a patient. For example, a bite block protects a patient from biting their tongue, lips, or cheeks, causing injury. More importantly, a bite block prevents a patient from biting down and compressing the pliable endotracheal tube. If an endotracheal tube is compressed during a surgical procedure or during the course of healing, it will result in an airway obstruction. As the patient tries to inhale while the endotracheal tube is obstructed, flash pulmonary edema (where fluid is pulled into the lungs from other adjacent compartments) may result. Flash pulmonary edema is a serious complication that results in prolonged need for intubation, possible ICU admission, longer hospital admission, and increased healthcare costs.

There are many inherent problems in the bite blocks found in the art, which can cause frustration for a user and, potentially, injury to a patient. For example, bite blocks come in a variety of shapes and materials and many healthcare providers improvise when it comes to using a bite block (e.g., gauze that is rolled and taped). A bite block that is too soft may not prevent the patient from biting down and compressing the endotracheal tube. On the other hand, bite blocks created out of a hard material may prevent compression of the endotracheal tube, but cause damage to the patient's teeth. Other bite blocks may clip or attach to the endotracheal tube, but they remain in a fixed position and are unable to slide along the endotracheal tube providing protection for the endotracheal tube when inserted or removed from the patient. When a medical provider uses an endotracheal tube, it is almost always used in tandem with a bite block, which is a separate movable part that inundates the mouth with objects.

Accordingly, there exists a need for a bite block that can be adjusted longitudinally on the endotracheal tube, is removably attachable to the endotracheal tube, and that remains on the endotracheal tube during extubation. Further, there exists a need for a bite block that has enough structural integrity to prevent compression of the endotracheal tube, but soft enough to prevent damage to a patient's teeth.

Further, it can be a challenging process to insert the endotracheal tube into a patient because of the differing airways that may be experienced from patient to patient. There are many airway tools available to providers, but there are currently no endotracheal tubes that have the ability of controlling the distal tip of the tube during intubations with the ability of having an adjustable bite block. Current practice involves inserting a laryngoscopy into the patient's oropharynx and then a stylet into the endotracheal tube, which gives the tube a certain degree of bend that a user can only guess will approximate the angle needed for safe passage of the tube through the patient's airway.

However, as previously mentioned, no one airway is the same, and it is not uncommon that the initial bend of the distal tip of the endotracheal tube needs to be adjusted. This usually results in the user needing to remove the breathing tube that has been unsuccessfully placed, reshaping the angle of the tube by bending the stylet, reinserting a laryngoscopy blade into the oropharynx, and reattempting to successfully place the endotracheal tube into the trachea. Repeated laryngoscopy attempts are associated with increased swelling, risk of airway trauma, and airway difficulty with each unsuccessful pass of the endotracheal tube. Additionally, once the endotracheal tube is in place, the stylet is typically removed by operating room personnel, which, if not done properly, can result in the breathing tube becoming dislodged or trauma to patients' lips or teeth.

Accordingly, there is a need for an endotracheal tube having an adjustable distal tip that does not require a removable stylet. In addition, there is a need for an endotracheal tube that can cause less airway and lip trauma, real-time ability to make fine adjustments of the endotracheal tube tip as it passes through the oropharynx and into the trachea, and a reduction in waste from non-reusable stylets.

### SUMMARY OF EXAMPLE EMBODIMENTS

In one embodiment, an intubation apparatus comprises an endotracheal tube and a removably attachable bite block. The removably attachable bite block may have a hard, inner surface and a soft, outer surface. In one embodiment, the removable attachable bite block is slidably adjustable along the length of an endotracheal tube.

In one embodiment, a bite block comprises a locking mechanism, such as a locking insert. In one embodiment, the locking insert comprises a tube receiving aperture and a locking aperture, wherein the diameter of the tube receiving aperture is greater than the diameter of the locking aperture. In another embodiment, the locking mechanism comprises a clamp. In one embodiment, the locking mechanism comprises a twist and lock mechanism.

In one embodiment, a removably attachable bite block comprises a small tubing slit.

In one embodiment, an endotracheal tube comprises a non-movable bite block.

In one embodiment, a method of using an intubation apparatus comprises coupling a removably attachable bite block apparatus to an endotracheal tube for insertion into the airway of a patient. After the tube is advanced to the appropriate depth, the user may slide the removably attachable bite block to the desired position, which is the location of the patient's teeth. Once the removably attachable bite block is in the desired location, the user locks the removably attachable bite block in position using a locking insert. When the user desires to remove the endotracheal tube, the user will loosen/unlock the removably attachable bite block on the endotracheal tube and safely pull the endotracheal tube through the removably attachable bite block while the removably attachable bite block remains in the desired position. After the endotracheal tube is completely removed, it is safe to remove the removably attachable bite block.

In one embodiment, an adjustable endotracheal tube comprises a distal tip adjustment mechanism, which controls the movement of the distal tip, wires, and a bite block. The distal tip adjustment mechanism has the ability to make real-time adjustments of the distal tip's shape and direction.

### BRIEF DESCRIPTION OF THE DRAWINGS

FIG. 1 illustrates a perspective view of an endotracheal tube and a removably attachable bite block of an intubation apparatus;

FIG. 2 illustrates a bottom perspective view of a removably attachable bite block of an intubation apparatus;

FIG. 3 illustrates a top, side perspective view of a removably attachable bite block of an intubation apparatus;

FIG. 4 illustrates an exploded view of a removably attachable bite block of an intubation apparatus;

FIG. 5 illustrates a top plan view of a removably attachable bite block of an intubation apparatus;

FIG. 6 illustrates a top, side perspective view of a removably attachable bite block with a locking insert in a first, unlocked position;

FIG. 7 illustrates a top, side perspective view of a removably attachable bite block with a locking insert in a second, locked position;

FIG. 8 illustrates a side perspective view of a removably attachable bite block in a first location;

FIG. 9 illustrates a side perspective view of a removably attachable bite block in a second location; and

FIG. 10 illustrates a side perspective view of a removably attachable bite block in a third location.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As previously discussed, there exists a need for a bite block that can be adjusted longitudinally on the endotracheal tube, is removably attachable to the endotracheal tube, and that remains on the endotracheal tube during extubation. Further, there exists a need for a bite block that has enough structural integrity to prevent compression of the endotracheal tube, but soft enough to prevent damage to a patient's teeth. There is also a need for an endotracheal tube that can control the distal tip during intubation, and does not require a removable stylet. In addition, there is a need for an endotracheal tube that can cause less airway and lip trauma, real-time ability to make fine adjustments of the endotracheal tube tip as it passes through the oropharynx and into the trachea, and a reduction in waste from non-reusable stylets.

Bite blocks used in a majority of healthcare institutions have many shortcomings, such as failing to completely protect a patient from damage to teeth or, even worse, pulmonary edema. Typically, bite blocks are separate from endotracheal tubes, which leads to the patient's mouth be overwhelmed with medical devices. Additionally, separate bite blocks may be easily and unintentionally dislodged from the patient's mouth, placing unnecessary risks on the patient. For example, a bite block that is separate from the endotracheal tube could be easily dislodged during insertion or extubation of the endotracheal tube, making the patient vulnerable. Further, many bite blocks on the market do not have the structural integrity to prevent a patient from biting and compressing the endotracheal tube, which could lead to pulmonary edema. On the other hand, many bite blocks are too hard, which could cause damage to the patient's teeth.

The removably attachable bite block described herein generally comprises a hard, inner surface and a soft, outer surface. The removably attachable bite block may receive an endotracheal tube via a first and second aperture. Accordingly, the removably attachable bite block may be adjustable along the length of the endotracheal tube, which will prevent the patient from biting and compressing the endotracheal tube during insertion and extubation. It will be appreciated that the adjustable bite block may be customized to any user, whether a child or an adult. The removably attachable bite block may also be secured in place. In other words, depending on the patient, the removably attachable bite block may be adjusted to the correct position and locked in place. It will also be appreciated that the removably attachable bite block, described below, removes the risk of a patient compressing the endotracheal tube as well as preventing damage to a patient's teeth and mouth.

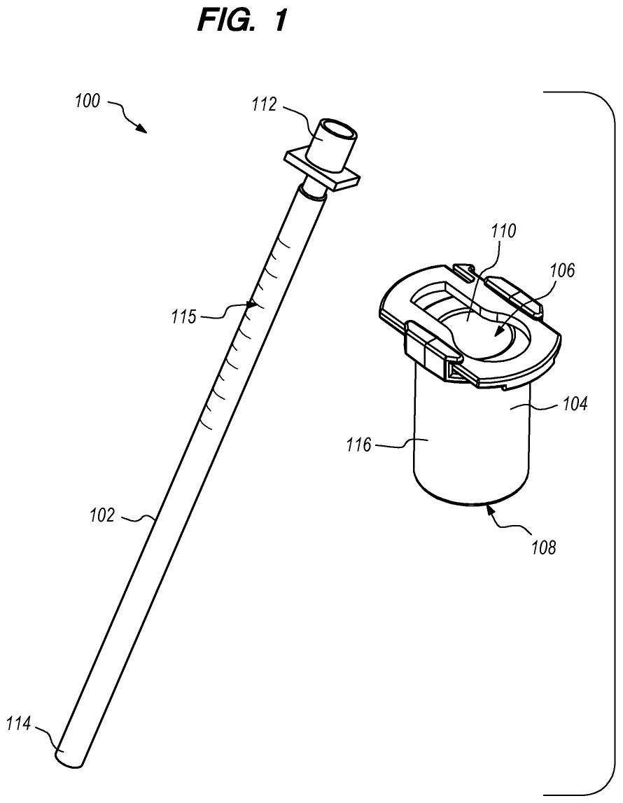

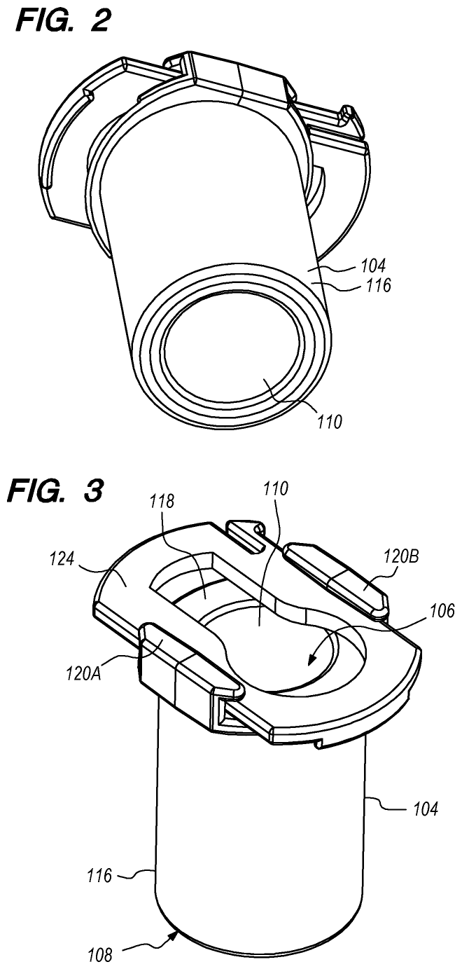

In one embodiment, as shown in FIGS. 1-2, an intubation apparatus 100 comprises an endotracheal tube 102 and a removably attachable bite block 104 that may be adjustable on the endotracheal tube 102. The removably attachable bite block 104 has a first aperture 106 and a second aperture 108 and an inner surface 110, which may be cylindrical, to receive and slide on an endotracheal tube 102 from a second end 114 to a first end 112. The removably attachable bite block 104 may be preassembled on the endotracheal tube 102 in a position that a user should expect to secure it to the tube 102, which can assist a user in knowing the depth of insertion; however, a user can slide the bite block 104 into other positions as needed. Further, the bite block 104 may be a separate accessory, removably attachable to any number of endotracheal tubes.

The removably attachable bite block 104 further comprises a soft outer surface 116 coupled to the inner surface 110. The soft outer surface 116 can be a silicone gel, rubber coating, or other suitable material for preventing damage to a patient's teeth. In some embodiments, the soft outer surface 116 may be removably attachable from the inner surface 110. While silicones or rubbers were used as an example, the removably attachable bite block 104 is not so limited. Accordingly, other materials that provide cushion without damaging a user's teeth may be used. Many bite blocks found in the prior art are made from a hard material, such as plastic, without having a soft outer surface. This has led to many patients damaging their teeth when biting down on the bite block. It will be appreciated that the soft outer surface 116 of the removably attachable bite block 104 can prevent damage to a patient's teeth, which is a significant improvement over the prior art.

The inner surface 110 of the removably attachable bite block 104 may be in direct contact with the endotracheal tube 102 and made of a hard material (e.g., PVC) that can withstand the typical bite force range of a patient, which is usually between 450 N and 1221 N. It is necessary that the bite block 104 be strong enough to prevent the endotracheal tube 102 from being compressed, which could cause injury to a patient. While PVC was provided as an example, other materials that can withstand the bite force range, such as metals, carbon fibers, and other suitable materials, may be used without departing herefrom. In one embodiment, the removably attachable bite block 104 may be a transparent material so that the user can see depth measurements 115 on an endotracheal tube. However, transparency is not required and other markers on the endotracheal tube may be used for depth measurements.

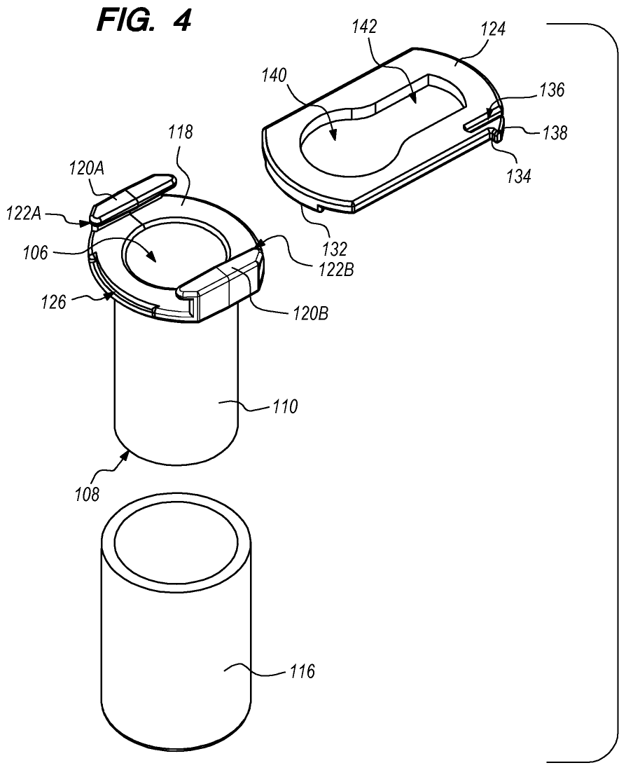

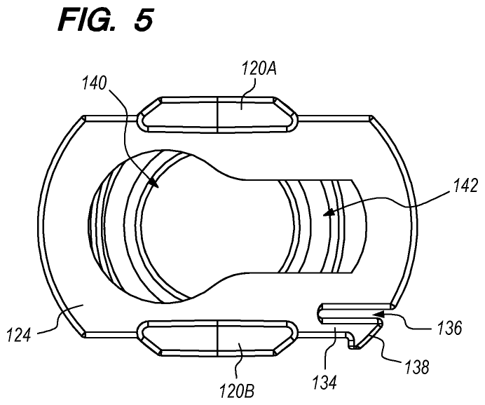

Referring to FIGS. 3-5, the hard inner surface 110 further comprises a locking platform 118 with a first finger 120A and a second finger 120B. The first and second fingers 120A, 120B extend vertically from the locking platform 118 creating a first channel 122A and a second channel 122B to receive a locking insert 124 therebetween. Further, the locking platform 118 may comprise a recessed portion 126 to inhibit inadvertent removal of the locking insert 124 in a first direction. The locking platform 118, as shown, is circular in shape; however, the locking platform 118 is not limited to a circle and may be a square, rectangle, etc.

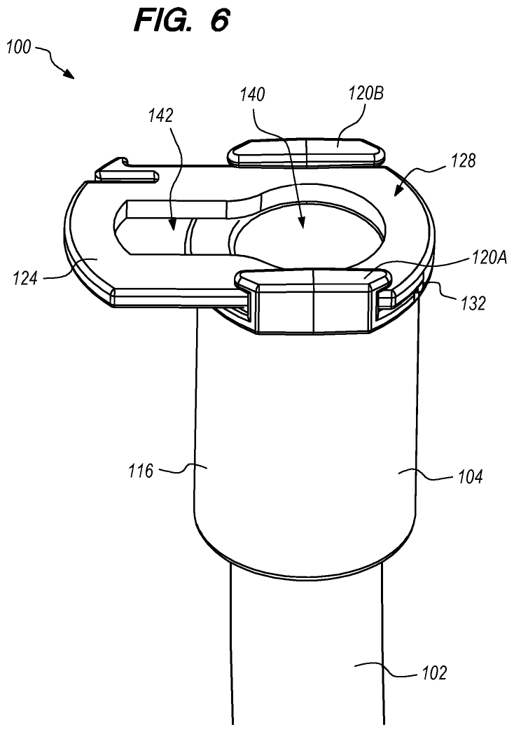

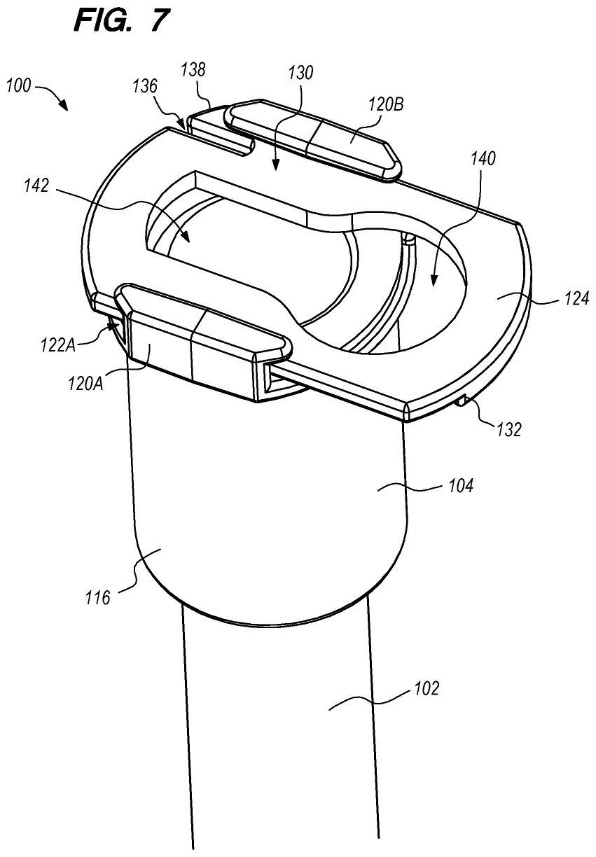

The locking insert 124 couples to the removably attachable bite block 104 via the first and second channels 122A, 122B on the locking platform 118. In other, words the locking insert 124 may be inserted in the first and second channels 122A, 122B so as to be adjusted from a first position 128 (FIG. 6) to a second position 130 (FIG. 7) in relation to the locking platform 118. When the locking insert 124 is inserted into the first and second channels 122A, 122B, a protrusion 132 on the locking insert 124 is received by the recessed portion 126 on the locking platform 118, thereby preventing its unintended withdrawal from the locking platform 118 in a first direction. On an opposite end of the protrusion 132 on the locking insert 124, there may be a locking lever 134, slit 136, and a hook 138. This allows the locking insert 124 to be inserted in a first direction into the channels 122A, 122B for coupling to the locking platform 118. In other words, the slit 136 allows the lever 134 and hook 138 to flex inward, allowing it to slide into the second channel 122B. Once passed the second finger 120B, the locking lever 134 returns to its normal position. As a result, when the locking insert 124 is pushed in a second direction, the hook 138 catches on the second finger 120B, preventing unintended withdrawal of the locking insert 124 in a second direction. If removal is desired, the locking lever and hook 138 may be compressed inward by a user, allowing the hook 138 to pass by the second finger 120B for removal. In addition, the locking lever 134 and hook 138 may be positioned on the corner of a patient's mouth to aid in keeping the bite block positioned. For example, when the desired depth is determined, the locking lever 134 may be hooked onto the corner of the patient's mouth, thereby securing the intubation apparatus 100 in the desired position.

As shown in FIGS. 6-7, the locking insert 124 has a tube receiving aperture 140 that may be aligned with the first and second apertures 106, 108 at the first position 128 (unlocked position shown in FIG. 6). The first position 128 allows the removably attachable bite block 104 to be positioned on and adjusted along the length of the endotracheal tube 102. At the second position 130 (FIG. 7), when a locking aperture 142 on the locking insert 124 is aligned with the first and second apertures 106, 108, the removably attachable bite block 104 is secured to the endotracheal tube 102. A simple sliding of the locking insert 124 prevents or allows movement of the removably attachable bite block 104.

As will be understood, the locking aperture 142 has a diameter that is slightly less than the outer diameter of the endotracheal tube 102. As a result, when the endotracheal tube is within the locking aperture 142 (e.g., at the second position 130), the locking insert 124 and the removably attachable bite block 104 is locked in place by compressive force. When the bite block 104 needs to move locations in relation to the endotracheal tube 102, the locking insert 124 may be forced or slid by a user so that the endotracheal tube 102 enters the first position 128 when the tube receiving aperture 140 is aligned with the first and second apertures 106, 108, which has a greater diameter than the locking aperture 142. As a result of the greater diameter, the endotracheal tube 102 is able to slide within the tube receiving aperture 140, allowing a practitioner the ability to adjust the position of the endotracheal tube 102 within a user while maintaining the bite block 104 in position. This also allows extubation without moving the bite block 104 from the desired position.

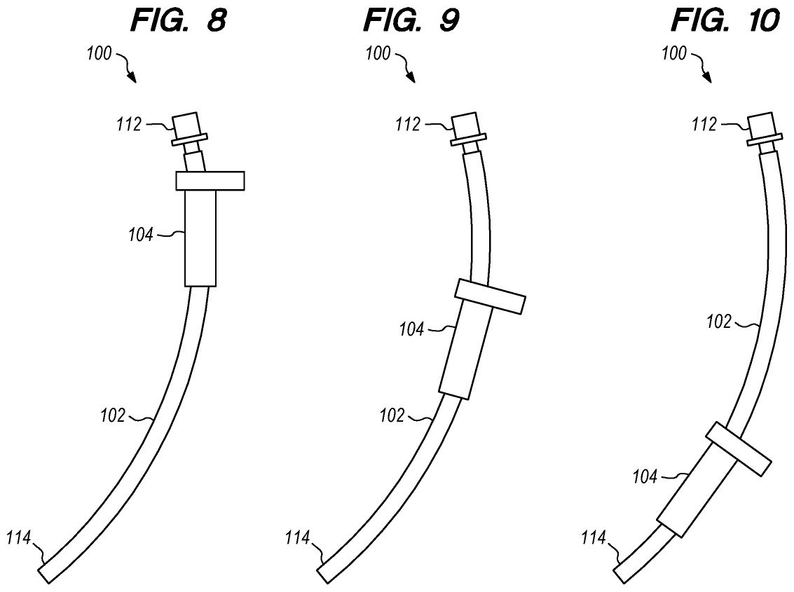

FIGS. 8-10 illustrate sliding the removably attachable bite block 104 in relation to the endotracheal tube 102. While the locking mechanism is illustrated as a slide and lock mechanism (e.g., locking insert 124), the locking mechanism can also be a gripping, pinching, or clamping mechanism, or other device for locking the position of the endotracheal tube. For example, rather than having a tube receiving aperture 140 and a locking aperture 142, a single aperture may exist that is hingedly openable using spring-controlled flanges. Other embodiments may utilize a twist and lock arrangement or other method of securing the bite block 104 in position, and such alterations are contemplated and do not depart herefrom.

The removably attachable bite block 104 may be of varying lengths depending on the size of the endotracheal tube 102, with the optimal length being, in some embodiments, between 3.0-5.0 cm. For example, when using a large stylet, it can be difficult to remove the stylet from within the bite block 104, especially if there is a significant bend in the stylet. It will be appreciated that reducing the length of the bite block may allow for easier and quicker removal of the stylet. The following, non-limiting, ranges illustrate example lengths of the removably attachable bite block 104 for a specific sized endotracheal tube: (1) For endotracheal tube sizes greater than 7.0 mm in internal diameter, the removably attachable bite block 104 may be approximately 4.5-5.0 cm in length; (2) For endotracheal tube sizes 5.5-6.5 mm in inside diameter, the removably attachable bite block 104 may be approximately 3.5-4.0 cm in length; and (3) For endotracheal tube sizes having a 5.0 mm inside diameter and smaller, the removably attachable bite block 104 may be approximately 3.0 cm in length. It will be appreciated to those in the art that a removably attachable bite block 104 of varying sizes will be able to accommodate all sizes of stylets, and that while ranges were provided above, these are exemplary only and variations are contemplated herein.

In one embodiment, the removably attachable bite block 104 may be curved to minimize resistance when a stylet is inserted or removed. In an alternate embodiment, a removably attachable bite block comprises a first end and a second end of the same inner diameter. However, the body between the first and second end has a slightly larger inner diameter to accommodate all sizes of curved stylets. In addition, the first and second ends are the coupling areas of the removably attachable bite block. When the body is slightly larger in diameter between the first and second end, the removably attachable bite block 104 can be the same length, without varying, for all sizes of endotracheal tubes.

As appreciated, the removably attachable bite block 104 can be slid with accuracy to a specific position and be locked into place with the locking insert 124. In addition, the locking insert 124 may be color coded. For example, the area around the locking aperture 142 may be red to signify a locked position, while the area around the tube aperture 140 may be green. As a user needs to reposition the bite block 104 using the locking insert 124, they will know whether it is locked by the position of the tube 102 in relation to the color of the locking insert 124. In one example, the removably attachable bite block 104 and locking insert 124 may be preassembled on an endotracheal tube 102 and locked into a predetermined position. Frequently, respiratory therapists, ER doctors, or critical care intensivists focus their attention on getting the endotracheal tube through the vocal cords and into the airway. After the endotracheal tube is advanced through the vocal cords, there is a tendency for these providers to over-insert the tube, which can result in mainstem intubations (where the tube is advanced so far that only one lung is being ventilated). This is problematic for many reasons; however, with the bite block preassembled onto the endotracheal tube where the teeth are most likely to make contact, this allows these providers to recognize the most likely depth of insertion needed for proper endotracheal tube placement. As a result, patients are properly intubated faster and with less injury or risk of injury to the patient.

The combination of the removably attachable bite block 104 and locking insert 124 solves issues in the prior art. For example, without the ability to secure the removably attachable bite block, a user would have to secure the bite block in place by other means, such as tape. If the tube needs to be adjusted or removed, the tape must either be removed from the bite block or the bite block is removed during extubation, which could be dangerous. In contrast FIGS. 8-10 illustrate how the bite block 104 and locking insert 124 can vary in position in relation to the endotracheal tube 102. This protects the endotracheal tube 102 from being compressed by the patient and, ultimately, protects the patient from serious injury.

Therefore, in one embodiment, a method of use comprises a physician positioning the bite block 104 and locking insert 124 in a desired position (e.g., bite block 104 interposed between the top and bottom teeth of a patient). The physician then inserts the endotracheal tube 102 into a patient. Because the endotracheal tube 102 is in the tube aperture 140 of the locking insert 124, the bite block 104 remains between the teeth while the tube 102 slides into position. Once positioned, the physician pushes the locking insert 124 so that the tube 102 enters the locking aperture 142 (i.e., second, locked position), thereby prohibiting the tube 102 from sliding due to the compressive forces. For extubation, the physician again actuates the locking insert 124, unlocking it, so that the tube 102 is slidable therein (e.g., positioned within the tube aperture 140 or first position). The tube 102 may then be removed from a patient while the bite block 104 remains interposed between the teeth. As a result, dangerous conditions are minimized or eliminated for the patient. Further, because the bite block 104 has a soft outer layer, the patient's teeth are not damaged.

In one embodiment, an intubation apparatus may comprise a small tubing slit (a line section removed lengthwise) along the side from a first aperture of the bite block to a second aperture of the bite block. General endotracheal tubes comprise a first end and a second end. The first end remains external to the mouth while the second end is inserted into a patient's mouth. The second end typically comprises a balloon/cuff that is inflated and deflated by using a small balloon tubing and a syringe. The small balloon tubing runs through the inside of the endotracheal tube from the balloon to where it exits. Some catheters exit the middle portion of the tube, while others, like the nasal endotracheal tubes, exit the first end. The slit on the removably attachable bite block allows endotracheal tubes having small balloon tubing to be utilized therewith. When extubation occurs with one of these tubes, the small tubing slit allows the endotracheal tube to be completely removed without catching on the small balloon tubing or balloon. The removably attachable bite block with the small tubing slit can allow any type of intubation tubing to slide through the first aperture of the bite block and the second aperture of the bite block, which can provide protection for the patient.

In one embodiment, not shown, an endotracheal tube comprises a non-movable bite block. The non-movable bite block is placed on the endotracheal tube at an optimal position. The optimal position can vary from patient to patient; however, the non-movable bite block in a length from 3-5 cm long can be fixed in a calculated position determined by the target depth range of the specific endotracheal tube that will work for most patients. The bite blocks in the prior art are not permanently coupled to the endotracheal tube. Accordingly, it may be a hassle to deal with a bite block that is moveable and not easily secured to the endotracheal tube. Therefore, with the non-movable bite block, there are fewer movable parts to worry about while ensuring the safety of the patient.

In one embodiment, an adjustable endotracheal tube comprises a distal tip adjustment mechanism, which controls the movement of the distal tip, wires, and a bite block. The distal tip adjustment mechanism has the ability to make real-time adjustments of the distal tip's shape and direction. The distal tip adjustment mechanism may be, but is not limited to, a sliding switch, lever, a roller (e.g., dial), or button that through an actuating system (e.g., pulley, lever, rotational) the distal tip may be manipulated. To actuate the distal tip adjustment, a user would move, for example, the roller which would move the distal tip to the desired position. The distal tip adjustment mechanism may further comprise a ribbed or textured surface so as to provide for easy manipulation.

Because the wires couple the distal tip adjustment mechanism to the distal tip, when the distal tip adjustment mechanism is actuated, the distal tip is likewise maneuvered. The wires can be made from any suitable material, including nylon, metal, etc. It will be appreciated that the distal tip adjustment mechanism may be slidable and coupled to a bite block. Having a slidable distal tip adjustment mechanism, that also acts as a bite block, allows the user to not only adjust the distal tip for ease of insertion, but protect a patient by being able to protect the endotracheal tube. While one embodiment may have a distal tip adjustment mechanism that acts as a bite block, the distal tip adjustment mechanism can be independent from the bite block. The distal tip adjustment mechanism may also be transparent, allowing the depth measurements on the endotracheal tube to remain visible. In an alternate embodiment, the adjustable endotracheal tube comprises a removably attachable bite block.

The adjustable endotracheal tube does not require the use of a stylet. Using stylets in the prior art can cause a lot of problems when it comes to inserting the endotracheal tube. For example, repeated endotracheal and laryngoscopy blade removal from the oropharynx to adjust the stylet shape during intubation can cause lip and airway trauma. In contrast, when using the adjustable endotracheal tube disclosed herein, there is no need for repeated endotracheal and laryngoscopy blade removal from the oropharynx to adjust stylet shape during intubation, which decreases the time to airway securement. Also, no stylet would need to be removed, thus also reducing the risk of tube dislodgement and lip trauma.

Therefore, it is appreciated from the foregoing disclosure that the adjustably removable bite block solves the need for a bite block that reduces or eliminates the risk of a patient biting down on the endotracheal tube during intubation and extubation. Further, the ability to control the distal tip without the need of a stylet reduces or eliminates injury to a patient as well.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. An intubation apparatus comprising:
an endotracheal tube;
a removably attachable bite block slidable along an entire length of the endotracheal tube from a first end to a second end, the removably attachable bite block comprising:
a first aperture and a second aperture,
a locking platform,
a locking insert coupleable to and slidable on the locking platform, the locking insert comprising a locking aperture and a tube receiving aperture;
wherein at a first position, when the tube receiving aperture is aligned with the first and second apertures, the removably attachable bite block is in an unlocked position;
wherein at a second position, when the locking aperture is aligned with the first and second apertures, the removably attachable bite block is in a locked position.

2. The intubation apparatus of claim 1, wherein the removably attachable bite block is cylindrical.

3. The intubation apparatus of claim 1, wherein the removably attachable bite block comprises a hard, inner surface.

4. The intubation apparatus of claim 1, wherein the removably attachable bite block comprises a soft, outer surface.

5. The intubation apparatus of claim 1, wherein the endotracheal tube comprises depth measurements.

6. The intubation apparatus of claim 5, wherein the removably attachable bite block is transparent to see the depth measurements.

7. The intubation apparatus of claim 1, wherein the tube receiving aperture is greater in diameter than the locking aperture.

8. An intubation apparatus comprising:
an endotracheal tube;
a removably attachable bite block slidable along an entire length of the endotracheal tube from a first end to a second end, the removably attachable bite block comprising:
a first aperture and a second aperture,
an inner surface that contacts the endotracheal tube,
an outer soft surface for contact with a user's oral cavity,
a locking platform comprising a first finger and a second finger,
a locking insert coupleable to and slidable under the first finger and the second finger on the locking platform, the locking insert comprising a locking aperture and a tube receiving aperture;
wherein at a first position, when the tube receiving aperture is aligned with the first and second apertures, the removably attachable bite block is in an unlocked position, allowing the endotracheal tube to be removed therefrom;
wherein at a second position, when the locking aperture is aligned with the first and second apertures, the removably attachable bite block is in a locked position that prohibits movement of the endotracheal tube.

9. The intubation apparatus of claim 8, wherein the inner surface is a hard material.

10. The intubation apparatus of claim 8, wherein the outer surface is a soft material.

11. The intubation apparatus of claim 8, wherein the first and second fingers comprise a first and second channel.

12. The intubation apparatus of claim 8, wherein the locking platform comprises a recessed portion to receive a protrusion on the locking insert.

13. The intubation apparatus of claim 8, wherein the locking insert comprises a locking lever, a slit, and a hook.

14. A removably attachable bite block coupleable to an endotracheal tube, the removably attachable bite block comprising:
a removably attachable bite block slidable along an entire length of the endotracheal tube, the removably attachable bite block comprising a locking mechanism;
wherein when the locking mechanism is in a first position, the endotracheal tube is slidable therein; and
wherein when the locking mechanism is in a second position, the endotracheal tube is fixed in position in relation to the removably attachable bite block.

15. The intubation apparatus of claim 14, wherein the removably attachable bite block comprises a hard inner surface.

16. The intubation apparatus of claim 14, wherein the removably attachable bite block comprises a soft outer surface.
